(19) United States
(12) Patent Application Publication
(10) Pub. No.: US 2026/0153259 A1
(43) Pub. Date: Jun. 4, 2026

(54) INTELLIGENT NETWORKED EXHAUST FAN

(71) Applicant: Microjet Technology Co., Ltd., Hsinchu (TW)

(72) Inventors: Hao-Jan Mou, Hsinchu (TW); Chin-Chuan Wu, Hsinchu (TW); Chi-Feng Huang, Hsinchu (TW)

(73) Assignee: Microjet Technology Co., Ltd., Hsinchu (TW)

(21) Appl. No.: 19/003,658

(22) Filed: Dec. 27, 2024

(30) Foreign Application Priority Data
Nov. 29, 2024 (TW) 113146229

(51) Int. Cl. F24F 11/89; A61L 9/014; A61L 9/20; B01D 46/00; B01D 46/42; B01D 46/44; B01D 46/46; B01D 53/00; B01D 53/04; B01D 53/88; F24F 7/00; F24F 7/06; F24F 8/108; F24F 8/158; F24F 8/167; F24F 8/192; F24F 11/58; F24F 11/63; F24F 11/77; F24F 13/28; F24F 110/10; F24F 110/20; F24F 110/50

(52) U.S. Cl. CPC F24F 11/89; A61L 9/014; A61L 9/205; B01D 46/0028; B01D 46/0036; B01D 46/0045; B01D 46/429; B01D 46/442; B01D 46/448; B01D 46/46; B01D 53/007; B01D 53/04; B01D 53/88; F24F 7/065; F24F 8/108; F24F 8/158; F24F 8/167; F24F 8/192; F24F 11/58; F24F 11/63; F24F 11/77; F24F 13/28; A61L 2209/12; A61L 2209/14; B01D 2253/102; B01D 2255/802; B01D 2257/708; B01D 2259/804; B01D 2259/818; B01D 2273/30; B01D 2279/50; B01D 2279/65; F24F 2007/001; F24F 2110/10; F24F 2110/20; F24F 2110/50

(57) ABSTRACT

An intelligent networked exhaust fan is disclosed and includes a main body, an air guiding fan, a host driving controller and a gas detection module. The main body has an air guiding path. The air guiding fan is disposed in the air guiding path for guiding air to exhaust. The host driving controller controls activation operation of the fan and dynamically adjusts an operating frequency and an output air volume of the air guiding fan. The gas detection module is electrically connected to the host driving controller, and configured to detect air pollution to generate a detection data, and the detection data is transmitted to a networked cloud computing service device through IoT communication. The networked cloud computing service device real-timely controls the host driving controller to control the activation operation of the fan and dynamically adjust operating frequency and output air volume of the fan.

### CROSS-REFERENCE TO RELATED APPLICATION

This application claims priority to Taiwan Patent Application No. 113146229, filed on Nov. 29, 2024. The entire contents of the above-mentioned patent application are incorporated herein by reference for all purposes.

### FIELD OF THE INVENTION

The present disclosure relates to an air quality monitoring and purification technology, and more particularly to an intelligent networked exhaust fan capable of automatically detecting air quality and adjusting air volume to achieve air purification and air pollution control effects.

### BACKGROUND OF THE INVENTION

As the threat to health from indoor and outdoor air pollution increases, the demand for air quality monitoring and improvement is becoming increasingly intense. Most existing exhaust fans only have basic exhaust functions and cannot intelligently detect the source and concentration of air pollution. The inability to automatically adjust the wind speed or operating mode in response to changes in air pollution makes it difficult to effectively control indoor air quality. Therefore, the present disclosure aims to solve the problem and provides an intelligent networked exhaust fan capable of automatically detecting air quality and adjusting air volume to achieve air purification and air pollution control effects.

### SUMMARY OF THE INVENTION

One object of the present disclosure is to provide an intelligent networked exhaust fan, which includes a built-in gas detection module for detecting air pollution in real time. In addition, the gas detection module has cloud connection capabilities, which facilitates remote monitoring and operation by users. Moreover, the air pollution detection data is transmitted to a networked cloud computing service device through IoT communication (wireless communication or wired communication), the networked cloud computing service device intelligently selects a control command based on the collection and analysis of the detection data monitored real-time, and the control command is transmitted to the gas detection module to control activation operation of the air guiding fan and dynamically adjust the efficiency of operating frequency and output air volume of the air guiding fan. The air pollution is instantly removed, completely cleaned and discharged to the outdoor field to achieve air purification and air pollution control effects. Furthermore, unnecessary energy consumption is reduced, automation and optimization of operations are achieved, and the indoor ambient air quality. is regulated and maintained at the optimal temperature and humidity in a modern home environment.

In accordance with an aspect of the present disclosure, an intelligent networked exhaust fan is provided and includes a main body, at least one air guiding fan, a host driving controller and at least one gas detection module. The main body includes an air suction port and an air exhaust duct, wherein an air guiding path is disposed between the air suction port and the air exhaust duct. The at least one air guiding fan is disposed in the air guiding path of the main body for guiding air to exhaust. The host driving controller controls activation operation of the at least one air guiding fan, and dynamically adjusts an operating frequency and an output air volume of the at least one air guiding fan. The at least one gas detection module is electrically connected to the host driving controller, wherein the at least one gas detection module detects air pollution to generate a detection data, the detection data is transmitted to a networked cloud computing service device through IoT communication, and the networked cloud computing service device collects and analyzes the detection data in real time, intelligently selects and generates a control command according to the detection data, and transmits the control command to the at least one gas detection module for receiving, so that the host driving controller regulates the activation operation of the at least one air guiding fan, and dynamically adjusts the operating frequency and the output air volume of the at least one air guiding fan, to achieve real-time monitoring.

### BRIEF DESCRIPTION OF THE DRAWINGS

The above contents of the present disclosure will become more readily apparent to those ordinarily skilled in the art after reviewing the following detailed description and accompanying drawings, in which:

FIG. 1A is a schematic perspective view illustrating an intelligent networked exhaust fan according to an embodiment of the present disclosure;

FIG. 1B is a schematic cross-sectional view illustrating related components in the intelligent networked exhaust fan according to the embodiment of the present disclosure;

FIG. 2 is a schematic cross-sectional view illustrating the filtering component according to the embodiment of the present disclosure;

FIG. 3 is a schematic diagram illustrating the architecture of the gas detection module of the intelligent networked exhaust fan connected to the host driving controller and the networked cloud computing service device according to the embodiment of the present disclosure;

FIG. 4A is a schematic perspective view (1) illustrating the gas detection main part of the gas detection module according to the embodiment of the present disclosure;

FIG. 4B is another schematic perspective view (2) illustrating the gas detection main part of the gas detection module according to the embodiment of the present disclosure;

FIG. 5 is an exploded view illustrating the gas detection main part of the gas detection module according to the embodiment of the present disclosure;

FIG. 6A is a schematic perspective view (1) illustrating the base of the gas detection main part of the gas detection module according to the embodiment of the present disclosure;

FIG. 6B is another schematic perspective view (2) illustrating the base of the gas detection main part of the gas detection module according to the embodiment of the present disclosure;

FIG. 6C is a schematic view (3) illustrating the base combined with the laser component and the piezoelectric actuator separated from the base of the gas detection main part of the gas detection module according to the embodiment of the present disclosure;

FIG. 7 a schematic perspective view illustrating the combination of the piezoelectric actuator and the base of the gas detection main part of the gas detection module according to the embodiment of the present disclosure;

FIG. 8A is a schematic exploded view (1) illustrating the piezoelectric actuator of the gas detection main part of the gas detection module according to the embodiment of the present disclosure;

FIG. 8B is another schematic exploded view (2) illustrating the piezoelectric actuator of the gas detection main part of the gas detection module according to the embodiment of the present disclosure;

FIG. 9A is a schematic cross-sectional view (1) illustrating an action of the piezoelectric actuator of the gas detection main part of the gas detection module according to the embodiment of the present disclosure;

FIG. 9B is a schematic cross-sectional view (2) illustrating an action of the piezoelectric actuator of the gas detection main part of the gas detection module according to the embodiment of the present disclosure;

FIG. 9C is a schematic cross-sectional view (3) illustrating an action of the piezoelectric actuator of the gas detection main part of the gas detection module according to the embodiment of the present disclosure;

FIG. 10A is a schematic cross-sectional view (1) illustrating the gas detection main part of the gas detection module introducing gas according to the embodiment of the present disclosure;

FIG. 10B is a schematic cross-sectional view (2) illustrating the gas detection main part of the gas detection module detecting gas according to the embodiment of the present disclosure;

FIG. 10C is a schematic cross-sectional view (3) illustrating the gas detection main part of the gas detection module exhausting gas according to the embodiment of the present disclosure; and

FIG. 11 is a schematic diagram illustrating the architecture of the networked cloud computing service device according to the embodiment of the present disclosure.

### DETAILED DESCRIPTION OF THE PREFERRED EMBODIMENT

The present disclosure will now be described more specifically with reference to the following embodiments. It is to be noted that the following descriptions of preferred embodiments of this invention are presented herein for purpose of illustration and description only. It is not intended to be exhaustive or to be limited to the precise form disclosed.

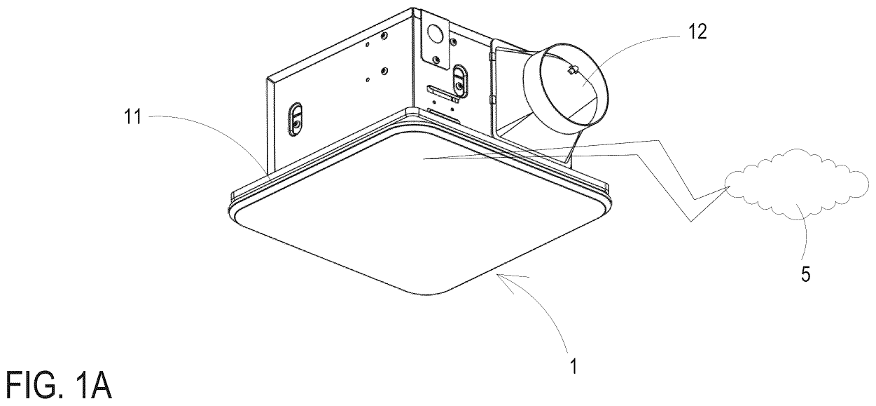

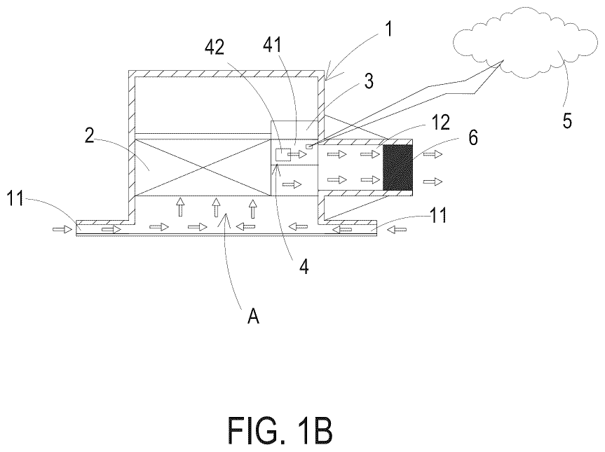

Please refer to FIG. 1A and FIG. 1B. The present disclosure provides an intelligent networked exhaust fan includes a main body 10, at least one air guiding fan 2, a host driving controller 3 and at least one gas detection module 4. It is worth noting that in the embodiment of the drawings, there is one air guiding fan 2 and one gas detection module 4, but the present disclosure is not limited thereto.

In the embodiment, the main body 1 includes an air suction port 11 and an air exhaust duct 12. An air guiding path A is disposed between the air suction port 11 and the air exhaust duct 12. The at least one air guiding fan 2 is disposed in the air guiding path A of the main body 1 for guiding air to exhaust. In the embodiment, the host driving controller 3 controls activation operation of the air guiding fan 2, and dynamically adjusting an operating frequency and an output air volume of the air guiding fan 2. Moreover, the gas detection module 4 is electrically connected to the host driving controller 3 for controlling. The gas detection module 4 is configured to detect humidity, temperature and air pollution to generate a detection data. The detection data is transmitted to a networked cloud computing service device 5 through IoT communication. The networked cloud computing service device 5 real-timely regulates the host driving controller 3 according to the detection data, so as to control the activation operation of the air guiding fan 2, and dynamically adjust the operating frequency and the output air volume of the air guiding fan 2. Therefore, as shown in FIG. 1B, the present disclosure provides an intelligent networked exhaust fan installed in an indoor field. When the air guiding fan 2 is enabled, the air pollution in the indoor field is inhaled through the air suction port 11, and then guided in the air guiding path A by the air guiding fan 2. The humidity, the temperature and the air pollution in the air are detected through the gas detection module 4, and a detection data is outputted. The air pollution is discharged to the outdoor field through the air exhaust duct 12. At the same time, the detection data detected by the gas detection module 4 is transmitted to a networked cloud computing service device 5 through IoT communication. The networked cloud computing service device 5 collects and analyzes the detection data monitored in real time, intelligently selects and generates a control command according to the detection data, and transmits the control command to the at least one gas detection module 4 for receiving, so that the host driving controller 3 regulates the activation operation of the at least one air guiding fan 2 in real time, and dynamically adjusts the operating frequency and the output air volume of the at least one air guiding fan 2, to achieve real-time monitoring. It can not only automatically detect the air quality and adjust air volume, but also instantly clean the air pollution completely and discharge the air pollution to the outdoor field, to achieve air purification and air pollution control effects. Furthermore, unnecessary energy consumption is reduced, automation and optimization of operations are achieved, and the indoor ambient air quality. is regulated and maintained at the optimal temperature and humidity in a modern home environment.

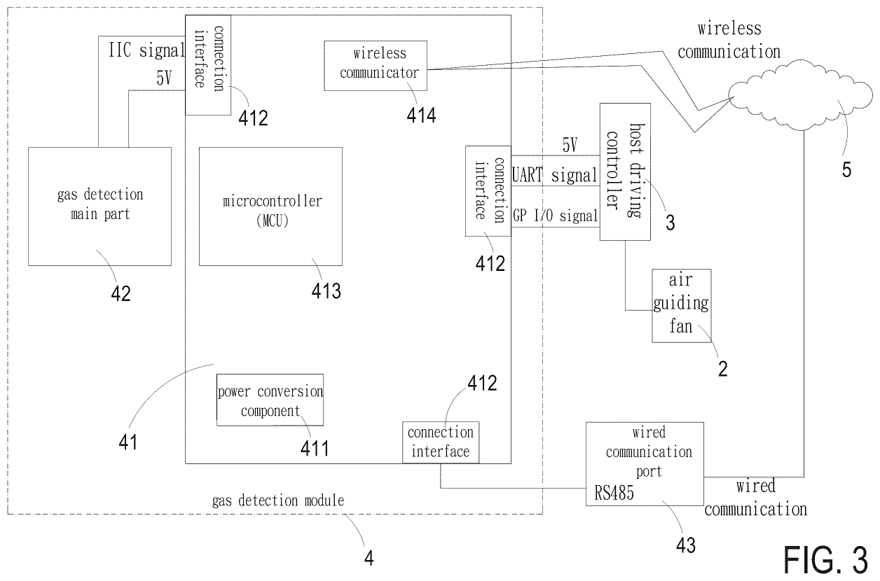

Please refer to FIG. 3. In the embodiment, the gas detection module 4 includes a controlling circuit board 41 and a gas detection main part 42. The gas detection main part 42 detects the humidity, the temperature and the air pollution to generate the detection data. The controlling circuit board 41 collects, calculates, analyzes and outputs the detection data to form a serial communication (IIC) signal for input, and the networked cloud computing service device 5 receives and analyzes the detection data in real time to output a Universal Asynchronous Transceiver and Transceiver (UART) signal and a General Purpose Input and Output (GP I/O) signal for the host driving controller 3. In the embodiment, the controlling circuit board 41 is embedded on the top surface of the main body 1 and is electrically connected to the host driving controller 3 for control. Moreover, the controlling circuit board 41 is signally connected to external components or devices through at least one connection interface 412. In the embodiment, the controlling circuit board 41 includes a plurality of connection interfaces 412, and the plurality of connection interfaces 412 are connected to the gas detection main part 42, the host driving controller 3 and a wired communication port 43, respectively for signal connection. Certainly, the present disclosure is not limited thereto. In an embodiment, the controlling circuit board 41 can select one connection interface 412 to connect with the gas detection main part 42, the host driving controller 3 and a wired communication port 43, respectively for signal connection. In the embodiment, the controlling circuit board 41 includes a power conversion component 411, a microcontroller (MCU) 413 and a wireless communicator 414. The power conversion component 411 provides DC voltage division modulation to output a required DC voltage. The required DC voltage is transmitted through at least one connection interface 412 to the gas detection main part 42 for actuation operation and to the host driving controller 3 for actuation operation. The microcontroller (MCU) 413 is connected to the gas detection main part 42 through the at least one connection interface 412 to form the serial communication (IIC) signal for input, so that the detection data is calculated and analyzed, and connected through the at least one connection interface 412 to output the Universal Asynchronous Transceiver and Transceiver (UART) signal and the General Purpose Input and Output (GP I/O) signal for regulation of the host driving controller 3. The wireless communicator 414 receives the detection data and transmits the detection data to the networked cloud computing service device 5 through external wireless communication. The networked cloud computing service device 5 collects, analyzes and monitors the detection data in real time and intelligently selects a control command, and the control command is received through the wireless communicator 414 and transmitted to the microcontroller (MCU) 413 to output the Universal Asynchronous Transceiver and Transceiver (UART) signal and the General Purpose Input and Output (GP I/O) signal for regulation of the host driving controller 3, so that the host driving controller 3 is regulated to control the activation operation of the at least one air guiding fan, 2 and dynamically adjust the operating frequency and the output air volume of the at least one air guiding fan 2. In an embodiment, the intelligent networked exhaust fan further includes a wired communication port 43. The wired communication port 43 is electrically connected to the controlling circuit board 41 through a connection interface 412 for external connection to a wired communication transmission. The detection data is received and transmitted to the networked cloud computing service device 5 through external wireless communication, the networked cloud computing service device 5 collects, analyzes and monitors the detection data in real time and intelligently selects a control command, and the control command is received through the wired communication port 43 and transmitted to the microcontroller (MCU) 413 to output the Universal Asynchronous Transceiver and Transceiver (UART) signal and the General Purpose Input and Output (GP I/O) signal for regulation of the host driving controller 3, so that the host driving controller 3 is regulated to control the activation operation of the at least one air guiding fan 2, and dynamically adjust the operating frequency and the output air volume of the at least one air guiding fan 2. Notably, in the embodiment, the wired communication port 43 is an RS485 port that communicates with the networked cloud computing service device 5 through a wired line connection.

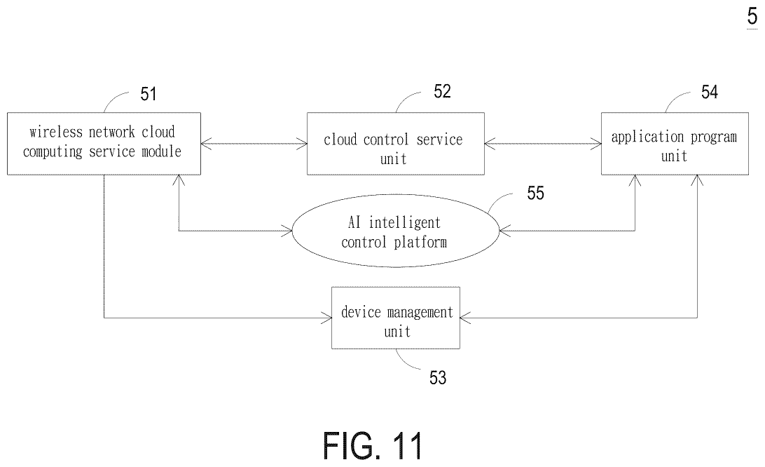

Moreover, as shown in FIG. 11, the networked cloud computing service device 5 includes a wireless network cloud computing service module 51, a cloud control service unit 52, a device management unit 53, an application program unit 54 and an AI intelligent control platform 55. In the embodiment, the wireless network cloud computing service module 51 receives the information of the air pollution data from the gas detection module 4 of the intelligent networked exhaust fan, and transmits the control commands. Moreover, the wireless network cloud computing service module 51 receives the information of the air pollution data and transmits the information to the cloud control service unit 52 to store and form the big data database of the air pollution data. An artificial intelligence calculation is implemented to determine the location of the air pollution through the air pollution database comparison, so that the control command is transmitted to the wireless network cloud computing service module 51, and then transmitted to the gas detection module 4 of the intelligent networked exhaust fan to control the actuation operation through the wireless network cloud computing service module 51. The device management unit 53 receives the communication information of the gas detection module 4 of the intelligent networked exhaust fan through the wireless network cloud computing service module 51 to manage the user login and device binding. It can also provide maintenance, management, automatic abnormal point detection, analysis, processing and improvement of intelligent networked exhaust fan. Management information, such as controlling inspection and measurement compliance with cleanliness requirements, customer demand feedback, and correction mechanisms for software and hardware technology improvements, is provided to the application unit for system control and management. Furthermore, the device management information can be provided to the application program unit 54 for system control and management, and the application program unit 54 can also display and inform the air pollution information obtained by the cloud control service unit 52. The user can know the real-time status of air pollution removal through the mobile phone or the communication device. Moreover, the user can control the operation of the indoor air cleaning network mechanism intelligent system through the application program unit 54 of the mobile phone or the communication device. In addition, the AI intelligent control platform 55 collects, analyzes and monitors the detection data in real time, intelligently selects and generates a control instruction, and transmits the control instruction to the at least one gas detection module 4 for receiving. In that, the host driving controller 3 is used to control the activation operation of the at least one air guiding fan 2, and dynamically adjust the operating frequency and the output air volume of the air guiding fan 2. That is to say, the control command issued by intelligent judgment is sent to the host driving controller 3 to control the activation operation of the air guiding fan 2 and dynamically adjust the operating frequency and the output air volume of the air guiding fan 2. As the gas detection data is greater than the safety detection value, the output air volume of the air guiding fan 2 is adjusted to be larger, and the air guide fan 2 is automatically started to strengthen the purification mode. As the gas detection data is close to the safety detection value, the output air volume of the air guiding fan 2 is adjusted to be smaller. According to the collection and analysis of real-time monitoring detection data, the operating frequency of the air guiding fan 2 is dynamically adjusted. It can be automatically switched to a low energy consumption mode, so as to reduce airflow noise. Even when the air quality in the indoor area is cleaned completely, the operation will be stopped to reduce unnecessary energy consumption.

From the above descriptions, the present disclosure provides an intelligent networked exhaust fan including the built-in gas detection module 4 with the cloud connection capabilities to be implemented in the intelligent system of indoor air purification network mechanism. All air pollution detection data can be uploaded to the networked cloud computing service device 5, so that the intelligent networked exhaust fan can use the networked cloud intelligent control of the gas detection module 4 to real-time monitor the activation state and dynamically adjust the efficiency of operating frequency and output air volume of the air guiding fan 2. It can not only automatically detect the air quality and adjust air volume, but also instantly clean the air pollution completely and discharge the air pollution to the outdoor field, to achieve air purification and air pollution control effects. Furthermore, unnecessary energy consumption is reduced, automation and optimization of operations are achieved, and the indoor ambient air quality. is regulated and maintained at the optimal temperature and humidity in a modern home environment.

After understanding the overall structure of the intelligent networked exhaust fan of the present disclosure, the detailed structure of the gas detection main part 42 of the gas detection module 4 will be described in detail below.

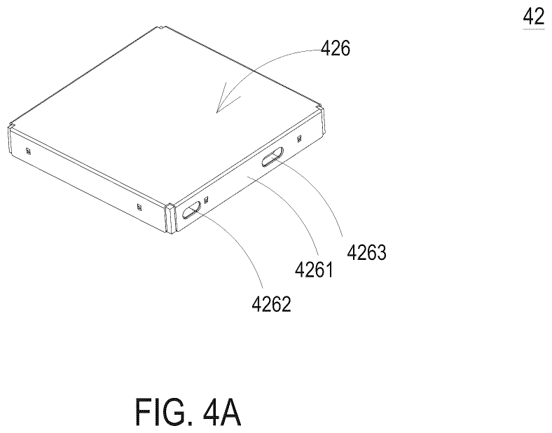

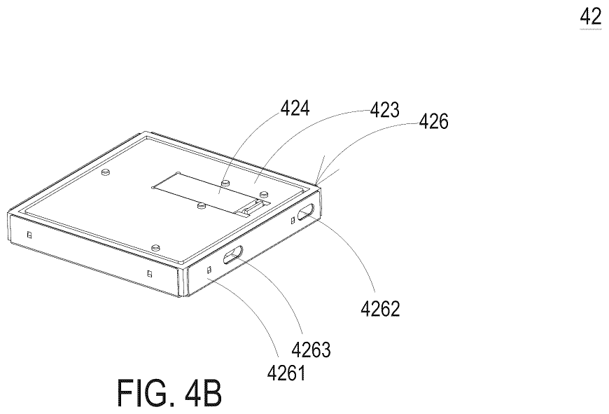

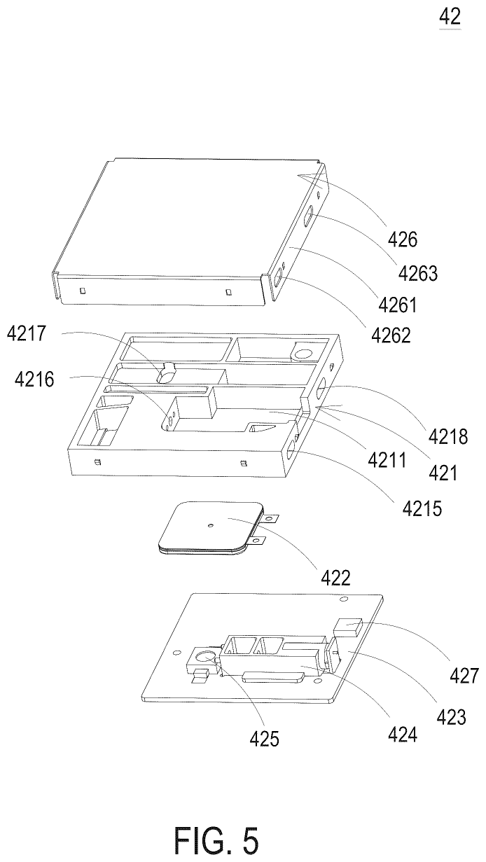

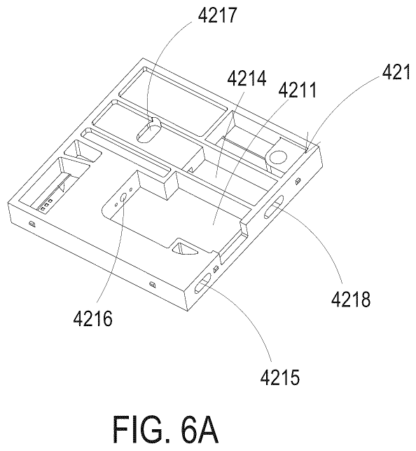

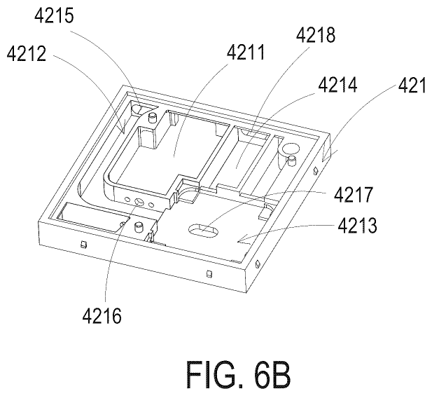

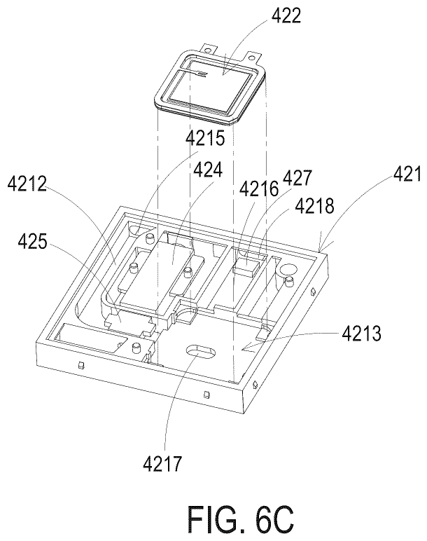

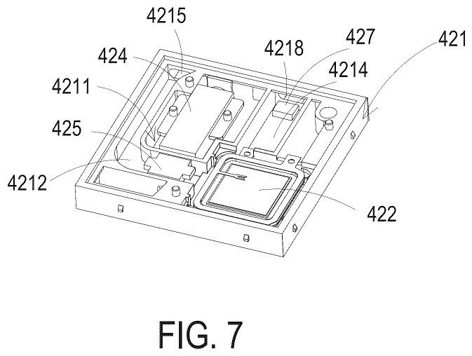

Please refer to FIG. 4A, FIG. 4B, FIG. 5, FIG. 6A to FIG. 6C and FIG. 7. In the embodiment, the gas detection main part 42 includes a base 421, a piezoelectric actuator 422, a driving circuit board 423, a laser component 424, a particulate sensor 425, an outer cover 426 and a gas sensor 427.

In the embodiment, the base 421 includes a laser loading region 4211, a gas-inlet groove 4212, a gas-guiding-component loading region 4213 and a gas-outlet groove 4214. The gas-inlet groove 4212 includes a gas-inlet 4215 and two lateral walls, the gas-inlet 4215 is in communication with an environment outside the base, and a transparent window 4216 is opened on the two lateral walls and is in communication with the laser loading region 4211. The gas-guiding-component loading region 4213 is in communication with the gas-inlet groove 4212, and a ventilation hole 4217 penetrates a bottom surface of the gas-guiding-component loading region 4213. The gas-outlet groove 4214 is in communication with the ventilation hole 4217, and a gas-outlet 4218 is disposed in the gas-outlet groove 4214. In the embodiment, the outer cover 426 covers the base 421, and includes a side plate 4261. The side plate 4261 has an inlet opening 4262 and an outlet opening 4263. The inlet opening 4262 is spatially corresponding to the gas-inlet 4215 of the base 421, and the outlet opening 4263 is spatially corresponding to the gas-outlet 4218 of the base 421.

In the embodiment, the laser component 424, the particulate sensor 425 and the gas sensor 427 are disposed on and electrically connected to the driving circuit board 423 and located within the base 421. In order to clearly describe and illustrate the positions of the laser component 424 and the particulate sensor 425 in the base 421, the driving circuit board 423 is intentionally omitted. The laser component 424 is accommodated in the laser loading region 4211 of the base 421, and the particulate sensor 425 is accommodated in the gas-inlet groove 4212 of the base 421 and is aligned to the laser component 424. In addition, the laser component 424 is spatially corresponding to the transparent window 4216, therefore, a light beam emitted by the laser component 424 passes through the transparent window 4216 and is irradiated into the gas-inlet groove 4212. A light beam path emitted from the laser component 424 passes through the transparent window 4216 and extends in an orthogonal direction perpendicular to the gas-inlet groove 4212. In the embodiment, a projecting light beam emitted from the laser component 424 passes through the transparent window 4216 and enters the gas-inlet groove 4212 to irradiate the suspended particles contained in the gas passing through the gas-inlet groove 4212. When the suspended particles contained in the gas are irradiated and generate scattered light spots, the scattered light spots are received and calculated by the particulate sensor 425 in the orthogonal direction to obtain the gas detection data. Notably, the laser component 424 emits a parallel light source, and the parallel light source passes through the transparent window 4216.

In the embodiment, the gas sensor 427 is positioned and accommodated in the gas-outlet groove 4214, so as to detect the air pollution introduced into the gas-outlet groove 4214. Preferably but not exclusively, the particulate sensor 425 detects suspended particulate and outputs the detection data. Moreover, the gas sensor 427 includes a volatile-organic-compound sensor, and the volatile-organic-compound sensor detects gas of carbon dioxide (CO₂) or volatile organic compounds (TVOC) to output the detection data. In an embodiment, the gas sensor 427 is a formaldehyde sensor, and the formaldehyde sensor detects gas of formaldehyde (HCHO) to output the detection data. In an embodiment, the gas sensor 427 is a bacteria sensor, and the bacteria sensor detects gas information of bacteria or fungi to output the detection data. In an embodiment, the gas sensor 427 is a virus sensor, and the virus sensor detects gas of virus to output the detection data. In an embodiment, the gas sensor 427 is a temperature and humidity sensor, and the temperature and humidity sensor detects the temperature and humidity in air to output the detection data.

Please refer to FIG. 6C and FIG. 7. In the embodiment, the piezoelectric actuator 422 is accommodated in the gas-guiding-component loading region 4213 of the base 421. In addition, the gas-guiding-component loading region 4213 of the base 421 is in fluid communication with the gas-inlet groove 4212. When the driving circuit board 423 is covered inside the base 421 and the outer cover 426 is covered outside the base 421, the inlet opening 4262 corresponds to the gas-inlet 4215 of the base 421 to collaboratively define an inlet path, and the outlet opening 4263 corresponds to the gas-outlet 4218 of the base 421 to collaboratively define an air outlet path. When the piezoelectric actuator 422 is enabled, the gas in the gas-inlet groove 4212 is inhaled by the piezoelectric actuator 422, so that the gas flows into the piezoelectric actuator 422, and is transported into the gas-outlet groove 4214 through the ventilation hole 4217 of the gas-guiding-component loading region 4213. Finally, when the gas enters the gas-outlet groove 4214, the piezoelectric actuator 422 continuously transports the gas from the gas inlet path into the gas-outlet groove 4214, and the gas in the gas-outlet groove 4214 is pushed to the gas outlet path and through the gas-outlet 4218 and the outlet opening 4263 to discharge to the outside, to achieve the gas transportation at high speed and in large quantities.

After understanding the above structural description of the gas detection main part 42, the detailed structure of the piezoelectric actuator 422 will be described in detail below.

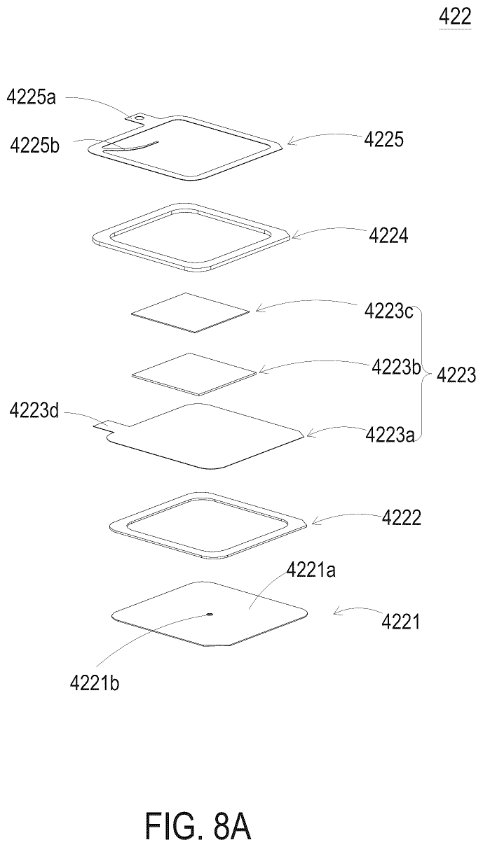

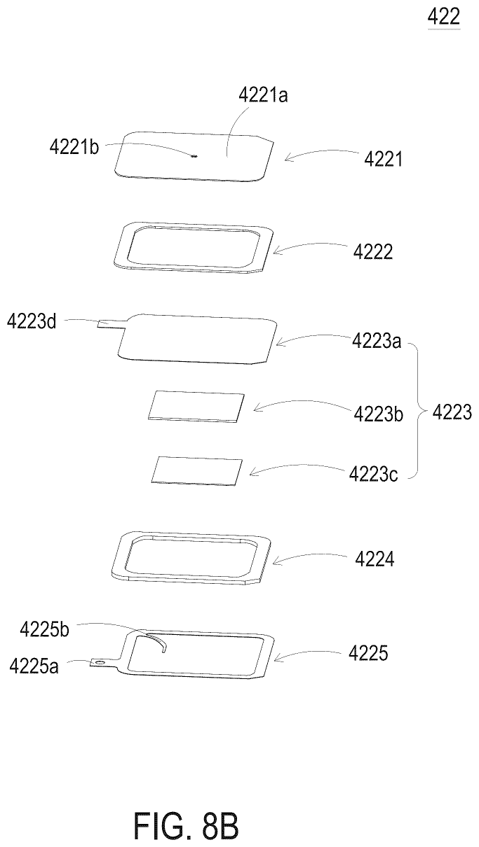

Please refer to FIG. 8A and FIG. 8B. In the embodiment, the piezoelectric actuator 422 includes a gas-injection plate 4221, a chamber frame 4222, an actuator element 4223, an insulation frame 4224 and a conductive frame 4225. In the embodiment, the gas-injection plate 4221 is made by a flexible material and includes a suspension plate 4221a and a hollow aperture 4221b. The suspension plate 4221a is a sheet structure and is permitted to undergo a bending deformation. Preferably but not exclusively, the shape and the size of the suspension plate 4221a are accommodated in the inner edge of the gas-guiding-component loading region 4215, but not limited thereto. The hollow aperture 4221b passes through a center of the suspension plate 4221a, so as to allow the gas to flow therethrough. Preferably but not exclusively, in the embodiment, the shape of the suspension plate 4221a is selected from the group consisting of a square, a circle, an ellipse, a triangle and a polygon, but not limited thereto.

In the embodiment, the chamber frame 4222 is carried and stacked on the gas-injection plate 4221. In addition, the shape of the chamber frame 4222 is corresponding to the gas-injection plate 4221. The actuator element 4223 is carried and stacked on the chamber frame 4222. A resonance chamber 4226 is collaboratively defined by the actuator element 4223, the chamber frame 4222 and the suspension plate 4221a and is formed between the actuator element 4223, the chamber frame 4222 and the suspension plate 4221a. The insulation frame 4224 is carried and stacked on the actuator element 4223 and the appearance of the insulation frame 4224 is similar to that of the chamber frame 4222. The conductive frame 4225 is carried and stacked on the insulation frame 4224, and the appearance of the conductive frame 4225 is similar to that of the insulation frame 4224. In addition, the conductive frame 4225 includes a conducting pin 4225a and a conducting electrode 4225b. The conducting pin 4225a is extended outwardly from an outer edge of the conductive frame 4225, and the conducting electrode 4225b is extended inwardly from an inner edge of the conductive frame 4225.

Moreover, the actuator element 4223 further includes a piezoelectric carrying plate 4223a, an adjusting resonance plate 4223b and a piezoelectric plate 4223c. The piezoelectric carrying plate 4223a is carried and stacked on the chamber frame 4222. The adjusting resonance plate 4223b is carried and stacked on the piezoelectric carrying plate 4223a. The piezoelectric plate 4223c is carried and stacked on the adjusting resonance plate 4223b. The adjusting resonance plate 4223b and the piezoelectric plate 4223c are accommodated in the insulation frame 4224. The conducting electrode 4225b of the conductive frame 4225 is electrically connected to the piezoelectric plate 4223c. In the embodiment, the piezoelectric carrying plate 4223a and the adjusting resonance plate 4223b are made by a conductive material. The piezoelectric carrying plate 4223a includes a piezoelectric pin 4223d. The piezoelectric pin 4223d and the conducting pin 4225a are electrically connected to a driving circuit (not shown) of the driving circuit board 423, so as to receive a driving signal, such as a driving frequency and a driving voltage. Through this structure, a circuit is formed by the piezoelectric pin 4223d, the piezoelectric carrying plate 4223a, the adjusting resonance plate 4223b, the piezoelectric plate 4223c, the conducting electrode 4225b, the conductive frame 4225 and the conducting pin 4225a for transmitting the driving signal. Moreover, the insulation frame 4224 is insulated between the conductive frame 4225 and the actuator element 4223, so as to avoid the occurrence of a short circuit. Thereby, the driving signal is transmitted to the piezoelectric plate 4223c. After receiving the driving signal such as the driving frequency and the driving voltage, the piezoelectric plate 4223c deforms due to the piezoelectric effect, and the piezoelectric carrying plate 4223a and the adjusting resonance plate 4223b are further driven to generate the bending deformation in the reciprocating manner.

Furthermore, in the embodiment, the adjusting resonance plate 4223b is located between the piezoelectric plate 4223c and the piezoelectric carrying plate 4223a and served as a cushion between the piezoelectric plate 4223c and the piezoelectric carrying plate 4223a. Thereby, the vibration frequency of the piezoelectric carrying plate 4223a is adjustable. Basically, the thickness of the adjusting resonance plate 4223b is greater than the thickness of the piezoelectric carrying plate 4223a, and the vibration frequency of the actuator element 4223 can be adjusted by adjusting the thickness of the adjusting resonance plate 4223b. In the embodiment, the gas-injection plate 4221, the chamber frame 4222, the actuator element 4223, the insulation frame 4224 and the conductive frame 4225 are stacked and positioned in the gas-guiding-component loading region 4213 sequentially, so that the piezoelectric actuator 422 is supported and positioned in the gas-guiding-component loading region 4213. A plurality of clearances 4221c are defined between the suspension plate 4221a of the gas-injection plate 4221 and an inner edge of the gas-guiding-component loading region 4213 for gas flowing therethrough.

In the embodiment, a flowing chamber 4227 is formed between the gas-injection plate 4221 and the bottom surface of the gas-guiding-component loading region 4213. The flowing chamber 4227 is in communication with the resonance chamber 4226 between the actuator element 4223, the gas-injection plate 4221 and the suspension plate 4221a. By controlling the vibration frequency of the gas in the resonance chamber 4226 to be close to the vibration frequency of the suspension plate 4221a, the Helmholtz resonance effect is generated between the resonance chamber 4226 and the suspension plate 4221a, so as to improve the efficiency of gas transportation. When the piezoelectric plate 4223c is moved away from the bottom surface of the gas-guiding-component loading region 4213, the suspension plate 4221a of the gas-injection plate 4221 is driven to move away from the bottom surface of the gas-guiding-component loading region 4213 by the piezoelectric plate 4223c. In that, the volume of the flowing chamber 4227 is expanded rapidly, the internal pressure of the flowing chamber 4227 is decreased to form a negative pressure, and the gas outside the piezoelectric actuator 422 is inhaled through the clearances 4221c and enters the resonance chamber 4226 through the hollow aperture 4221b. Consequently, the pressure in the resonance chamber 4226 is increased to generate a pressure gradient. When the suspension plate 4221a of the gas-injection plate 4221 is driven by the piezoelectric plate 4223c to move toward the bottom surface of the gas-guiding-component loading region 4213, the gas in the resonance chamber 4226 is discharged out rapidly through the hollow aperture 4221b, and the gas in the flowing chamber 4227 is compressed, thereby the converged gas is quickly and massively ejected out of the flowing chamber 4227 under the condition close to an ideal gas state of the Benulli's law, and transported to the ventilation hole 4217 of the gas-guiding-component loading region 4213.

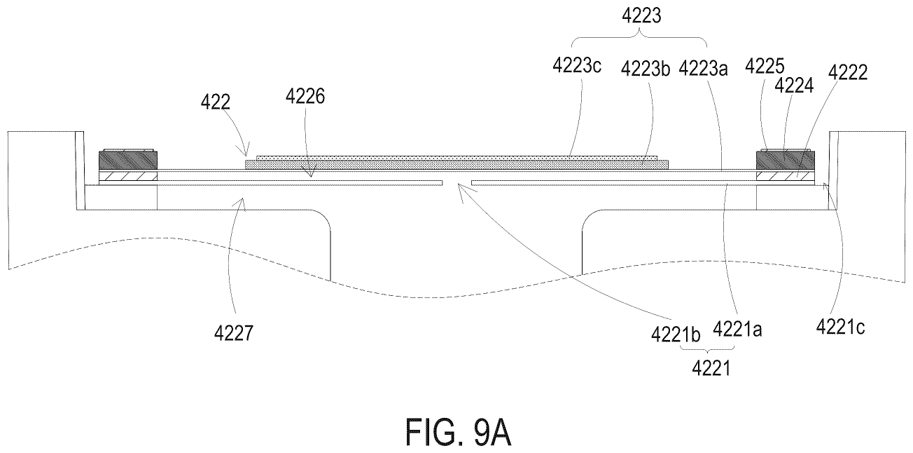

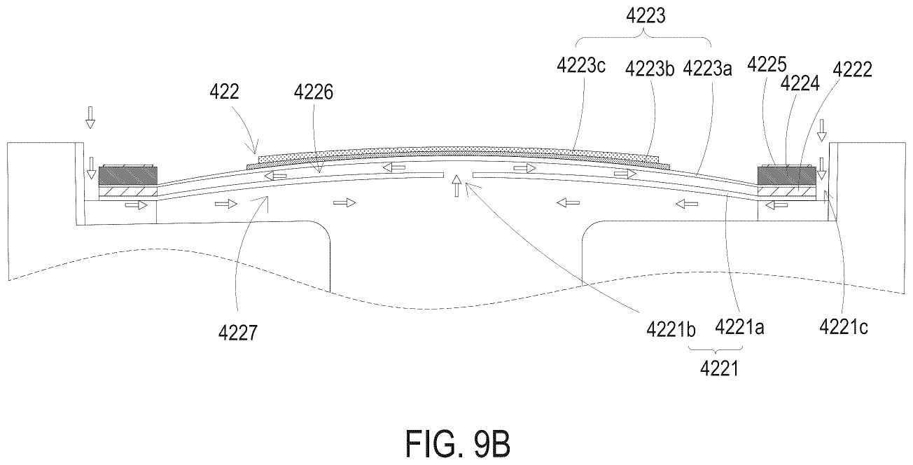

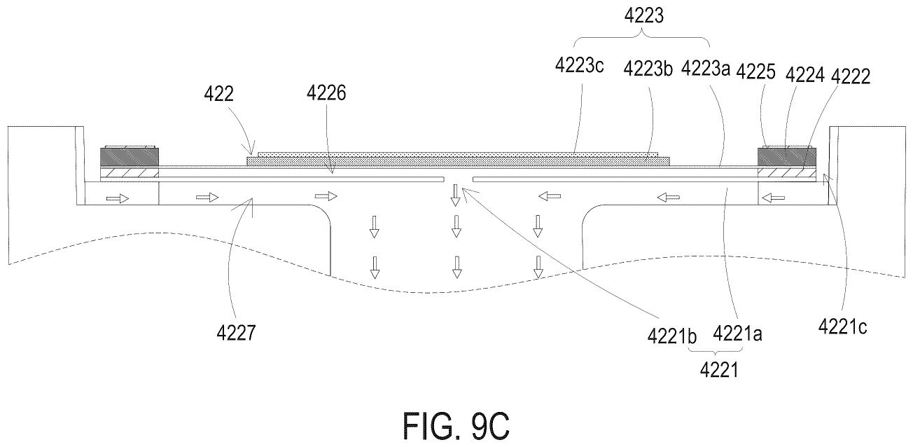

By repeating the above operation steps shown in FIG. 9B and FIG. 9C, the piezoelectric plate 4223c is driven to generate the bending deformation in a reciprocating manner. According to the principle of inertia, since the gas pressure inside the resonance chamber 4226 is lower than the equilibrium gas pressure after the converged gas is ejected out, the gas is introduced into the resonance chamber 4226 again. Moreover, the vibration frequency of the gas in the resonance chamber 4226 is controlled to be close to the vibration frequency of the piezoelectric plate 4223c, so as to generate the Helmholtz resonance effect to achieve the gas transportation at high speed and in large quantities.

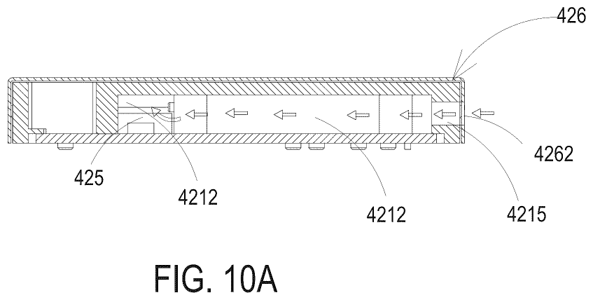

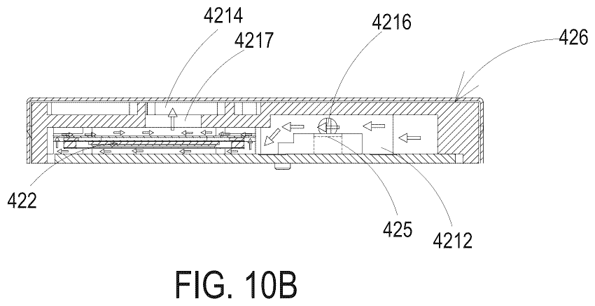

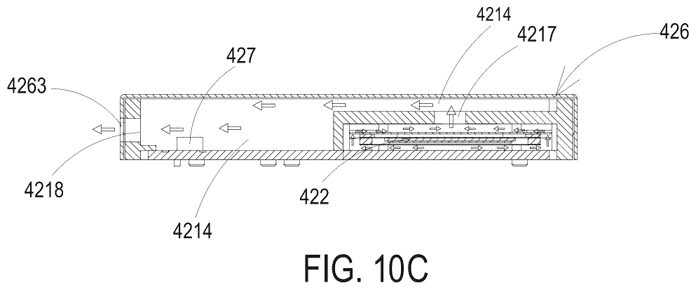

Please refer to FIG. 10A to FIG. 10C. The gas is inhaled through the gas-inlet 4262 on the outer cover 426, flows into the gas-inlet groove 4212 of the base 421 through the gas-inlet 4215, and is transported to the position of the particulate sensor 425. In addition, the piezoelectric actuator 422 is enabled continuously to inhale the gas into the inlet path, and facilitate the gas outside the gas detection module to be introduced rapidly, flow stably, and transported above the particulate sensor 425. At this time, a projecting light beam emitted from the laser component 424 passes through the transparent window 4216 to irritate the suspended particles contained in the gas flowing above the particulate sensor 425 in the gas-inlet groove 4212. When the suspended particles contained in the gas are irradiated and generate scattered light spots, the scattered light spots are received and calculated by the particulate sensor 425 for obtaining related information about the sizes and the concentration of the suspended particles contained in the gas. Moreover, the gas above the particulate sensor 425 is continuously driven and transported by the piezoelectric actuator 422, flows into the ventilation hole 4217 of the gas-guiding-component loading region 4213, and is transported to the gas-outlet groove 4214. At last, after the gas flows into the gas outlet groove 4214, the gas is continuously transported into the gas-outlet groove 4214 by the piezoelectric actuator 422, and thus the gas in the gas-outlet groove 4214 is pushed to discharge through the gas-outlet 4218a and the outlet opening 4263, to achieve the gas transportation at high speed and in large quantities.

Notably, in the above embodiment, the air pollution is at least one selected from the group consisting of particulate matter, carbon monoxide, carbon dioxide, ozone, sulfur dioxide, nitrogen dioxide, lead, total volatile organic compounds (TVOC), formaldehyde, bacteria, fungi, virus and a combination thereof. The IoT communication is a wireless communication for communicating with the networked cloud computing service device 5 through a wireless connection. Preferably but not exclusively, the wireless communication is one selected from the group consisting of a Wi-Fi communication, a Bluetooth communication, a radio frequency identification communication and a near field communication (NFC). Alternatively, the IoT communication is a wired communication for connecting and communicating with the networked cloud computing service device 5 through a wired line connection. In the embodiment, the air guiding fan 2 includes an exhaust volume greater than 20 m³/min, and a wind pressure greater than 30 mmAq. Preferably but not exclusively, the air guiding fan 2 includes the exhaust volume greater than 170 m³/min. Preferably, the air guiding fan 2 includes an optimal exhaust volume ranged from 170 m³/min to 500 m³/min.

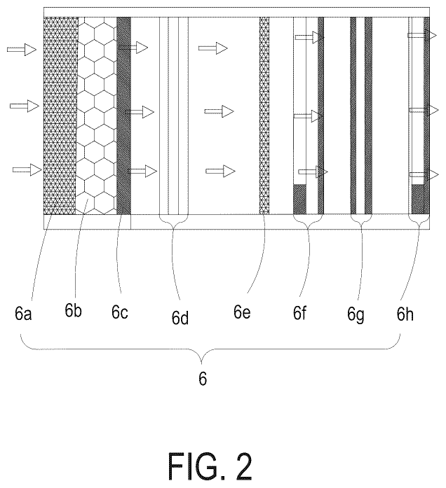

In the embodiment, the intelligent networked exhaust fan further includes a filtering component 6 disposed in the air guiding path A. Please refer to FIG. 2. The filtering component 6 of the present disclosure can be a combination of various implementation forms. Preferably but not exclusively, in an embodiment, the filtering component 6 is a filter screen 6a, and the filter screen 6a is a filter screen with a minimum filtration efficiency value (MREV) equal to or greater than level 8. In an embodiment, the filtering component 6 is a filter screen 6a, and the filter screen 6a is a high-efficiency particulate air (HEPA) filter screen grade, which is configured to absorb the chemical smoke, the bacteria, the dust particles and the pollen contained in the air pollution, so that the air pollution introduced is filtered and purified to achieve the effect of filtering and purification. Notably, in the present disclosure, the high-efficiency particulate air (HEPA) filter screen is equal to or greater than a high-efficiency particulate air filter (HEPA) grade 10, with a dust holding capacity greater than 12,000 mg. Alternatively, the filter screen 6a is a ULPA14 filter screen grade, so as to improve filtration efficiency and meet higher cleanliness requirements. In some specific embodiments, the filtering component 6 is further combined with physical or chemical materials to provide a sterilization effect for air pollution passing therethrough, and the airflow path direction of the air guiding fan 2 is the direction shown by the arrow. In an embodiment, the filtering component 6 is combined with a decomposition layer coated thereon to clean the air pollution through a chemical method of sterilization. Preferably but not exclusively, the decomposition layer is an activated carbon 6b for cleaning organic and inorganic substances in air pollution, and removing colored and odorous substances. Notably, the activated carbon 6b has a formaldehyde absorption capacity greater than 1500 mg. Moreover, in some embodiments, the filtering component 6 is combined with a light irradiation element to clean the air pollution through a chemical method of sterilization. Preferably but not exclusively, the light irradiation element is a photo-catalyst unit including a photo catalyst 6c and an ultraviolet lamp 6d for further improving the removal efficiency of pollutants and allergens in the air. When the photo catalyst 6c is irradiated by the ultraviolet lamp 6d, the light energy is converted into the chemical energy, thereby decomposes harmful gases and disinfects bacteria contained in the air pollution, so as to achieve the effects of filtering and purifying. Notably, in the present disclosure, the ultraviolet lamp 6 d has a power greater than 120 mW. In an embodiment, the light irradiation element is a photo-plasma unit including a nanometer irradiation tube 6e. When the introduced air pollution is irradiated by the nanometer irradiation tube 6e, the oxygen molecules and water molecules contained in the air pollution are decomposed into high oxidizing photo-plasma, and an ion flow capable of destroying organic molecules is generated. In that, volatile formaldehyde, volatile toluene and volatile organic compounds (VOC) contained in the air pollution are decomposed into water and carbon dioxide, so as to improve the removal efficiency of pollutants and allergens in the air, and achieve the effects of filtering and purifying. In some embodiments, the filtering component 6 is combined with a decomposition unit to clean the air pollution through a chemical method of sterilization. Preferably but not exclusively, the decomposition unit is a negative ion unit 6f with a dust collecting plate. It makes the suspended particles in the air pollution to carry with positive charge and adhered to the dust collecting plate carry with negative charges, so as to improve the removal efficiency of pollutants and allergens in the air, and achieve the effects of filtering and purifying. Preferably but not exclusively, the decomposition unit is a plasma ion unit 6g. The oxygen molecules and water molecules contained in the air pollution are decomposed into positive hydrogen ions (H⁺) and negative oxygen ions (O²⁻) by the plasma ion. The substances attached with water around the ions are adhered on the surface of viruses and bacteria and converted into OH radicals with extremely strong oxidizing power, thereby removing hydrogen (H) from the protein on the surface of viruses and bacteria, and thus decomposing (oxidizing) the protein, so as to improve the removal efficiency of pollutants and allergens in the air, and achieve the effects of filtering and purifying. Preferably but not exclusively, the decomposition unit is an electrostatic filtering unit 6h. The electrostatic force is used to capture and remove suspended particles (such as dust, pollen, bacteria and other pollutants) in the air.

From the above descriptions, the present disclosure provides an intelligent networked exhaust fan, which includes the built-in gas detection module 4 with the cloud connection capabilities to implement real-time monitoring and adjustment. The intelligent networked exhaust fan is further combined with the networked cloud computing service device 5 of the indoor air cleaning networked mechanism intelligent system, and has the following effects. For real-time monitoring and adjustment, the built-in gas detection module 4 can monitor the detection data of the humidity, temperature and the air pollution of indoor air in real time, and the detection data is transmitted to the Internet through IoT communication (wireless communication or wired communication). The cloud computing service device 5 regulates the activation operation of the air guiding fan 2 and dynamically adjusts the operating frequency and efficiency of the output air volume based on the collection and analysis of real-time monitoring detection data through the AI intelligent control platform 55. As the gas detection data is greater than the safety detection value, the output air volume of the air guiding fan 2 is adjusted to be larger, and the air guide fan 2 is automatically started to strengthen the purification mode. As the gas detection data is close to the safety detection value, the output air volume of the air guiding fan 2 is adjusted to be smaller. The gas detection module 4 is an intelligent cloud connection with cloud connection capabilities. All air pollution detection data can be uploaded to the networked cloud computing service device 5, and the users can remotely check the air quality of the indoor environment. The filtering component 6 has multiple filtration technologies, and the filtering component 6 disposed in the air guiding path A of the intelligent networked exhaust fan can be combined with activated carbon, high-efficiency filter, electrostatic filtration, photo catalyst unit, negative ion unit or plasma unit to achieve optimal filtration effects according to different pollution sources. When the indoor and outdoor environmental humidity is similar or the air quality reaches the standard, the networked cloud computing service device 5 will dynamically adjust the operating frequency of the air guiding fan 2 based on the collected and analyzed real-time monitoring detection data, automatically switch to a low energy consumption mode, and reduce the air volume noise. Even when the air quality in the indoor area is cleaned completely, the operation will be stopped to reduce unnecessary energy consumption. It allows multiple devices working together. If multiple intelligent networked exhaust fans are configured in the same indoor field space, the networked cloud computing service device 5 can adjust operations according to the air pollution detection data of each device to form a coordinated cleaning network to achieve the best air quality.

In summary, the present disclosure provides an intelligent networked exhaust fan, which includes a built-in gas detection module 4 for detecting air pollution in real time. In addition, the gas detection module 4 has cloud connection capabilities, which facilitates remote monitoring and operation by users. Moreover, the air pollution detection data is transmitted to a networked cloud computing service device 5 through IoT communication (wireless communication or wired communication), the networked cloud computing service device 5 intelligently selects a control command based on the collection and analysis of the detection data monitored real-time, and the control command is transmitted to the gas detection module 4 to control activation operation of the air guiding fan 2 and dynamically adjust the efficiency of operating frequency and output air volume of the air guiding fan 2. It can not only automatically detect the air quality and adjust air volume, but also instantly clean the air pollution completely and discharge the air pollution to the outdoor field, to achieve air purification and air pollution control effects. Furthermore, unnecessary energy consumption is reduced, automation and optimization of operations are achieved, and the indoor ambient air quality. is regulated and maintained at the optimal temperature and humidity in a modern home environment. The present disclosure includes the industrial applicability and the inventive steps.

What is claimed is:

1. An intelligent networked exhaust fan, comprising:
a main body comprising an air suction port and an air exhaust duct, wherein an air guiding path is disposed between the air suction port and the air exhaust duct;
at least one air guiding fan disposed in the air guiding path of the main body for guiding air to exhaust;
a host driving controller controlling activation operation of the at least one air guiding fan, and dynamically adjusting an operating frequency and an output air volume of the at least one air guiding fan; and
at least one gas detection module electrically connected to the host driving controller, wherein the at least one gas detection module detects air pollution to generate a detection data, the detection data is transmitted to a networked cloud computing service device through IoT communication, and the networked cloud computing service device collects and analyzes the detection data in real time, intelligently selects and generates a control command according to the detection data, and transmits the control command to the at least one gas detection module for receiving, so that the host driving controller regulates the activation operation of the at least one air guiding fan, and dynamically adjusts the operating frequency and the output air volume of the at least one air guiding fan, to achieve real-time monitoring.

2. The intelligent networked exhaust fan according to claim 1, wherein the air pollution is at least one selected from the group consisting of particulate matter, carbon monoxide, carbon dioxide, ozone, sulfur dioxide, nitrogen dioxide, lead, total volatile organic compounds (TVOC), formaldehyde, bacteria, fungi, virus and a combination thereof.

3. The intelligent networked exhaust fan according to claim 1, wherein the at least one air guiding fan includes an exhaust volume greater than 20 m³/min, and a wind pressure greater than 30 mmAq.

4. The intelligent networked exhaust fan according to claim 3, wherein the at least one air guiding fan includes the exhaust volume greater than 170 m³/min.

5. The intelligent networked exhaust fan according to claim 1, wherein the at least one air guiding fan includes an exhaust volume ranged from 170 m³/min to 500 m³/min.

6. The intelligent networked exhaust fan according to claim 1, wherein the IoT communication is a wireless communication for communicating with the networked cloud computing service device through a wireless connection, or a wired communication for communicating with the networked cloud computing service device through a wired connection, and the wireless communication is one selected from the group consisting of a Wi-Fi communication, a Bluetooth communication, a radio frequency identification communication and a near field communication (NFC).

7. The intelligent networked exhaust fan according to claim 1, wherein the networked cloud computing service device comprises an AI intelligent control platform, and the AI intelligent control platform collects, analyzes and monitors the detection data in real time, intelligently selects and generates a control instruction, and transmits the control instruction to the at least one gas detection module for receiving, wherein the host driving controller is used to control the activation operation of the at least one air guiding fan, and dynamically adjust the operating frequency and the output air volume of the air guiding fan.

8. The intelligent networked exhaust fan according to claim 1, wherein the gas detection module comprises a gas detection main part and a controlling circuit board, the gas detection main part detects humidity, the temperature and the air pollution to generate the detection data, the controlling circuit board collects, calculates, analyzes and outputs the detection data to form a serial communication (IIC) signal for input, and the networked cloud computing service device receives and analyzes the detection data in real time to output a Universal Asynchronous Transceiver and Transceiver (UART) signal and a General Purpose Input and Output (GP I/O) signal for the host driving controller.

9. The intelligent networked exhaust fan according to claim 8, wherein the gas detection main part comprises:
a base comprising a laser loading region, a gas-inlet groove, a gas-guiding-component loading region and a gas-outlet groove, wherein the gas-inlet groove comprises a gas-inlet and two lateral walls, the gas-inlet is in communication with an environment outside the base, and a transparent window is opened on the two lateral walls and is in communication with the laser loading region, the gas-guiding-component loading region is in communication with the gas-inlet groove, and a ventilation hole penetrates a bottom surface of the gas-guiding-component loading region, wherein the gas-outlet groove is in communication with the ventilation hole, and a gas-outlet is disposed in the gas-outlet groove;
a piezoelectric actuator accommodated in the gas-guiding-component loading region;
a driving circuit board covering and attached to the base;
a laser component positioned and disposed on the driving circuit board, electrically connected to the driving circuit board, and accommodated in the laser loading region, wherein a light beam path emitted from the laser component passes through the transparent window and extends in a direction perpendicular to the gas-inlet groove, thereby forming an orthogonal direction with the gas-inlet groove;
a particulate sensor positioned and disposed on the driving circuit board, electrically connected to the driving circuit board, and disposed at an orthogonal position where the gas-inlet groove intersects the light beam path of the laser component in the orthogonal direction, so that suspended particles contained in the air pollution passing through the gas-inlet groove and irradiated by a projecting light beam emitted from the laser component are detected;
at least one gas sensor positioned and disposed on the driving circuit board, electrically connected to the driving circuit board, and accommodated in the gas-outlet groove, so as to detect the air pollution introduced into the gas-outlet groove; and
an outer cover covering the base and comprising a side plate, wherein the side plate has an inlet opening and an outlet opening, the inlet opening is spatially corresponding to the gas-inlet of the base, and the outlet opening is spatially corresponding to the gas-outlet of the base;
wherein the outer cover covers the base, and the driving circuit board is attached to the base, thereby an inlet path is defined by the gas-inlet groove, and an outlet path is defined by the gas-outlet groove, so that the air pollution is inhaled from the environment outside the base by the piezoelectric actuator, transported into the inlet path defined by the gas-inlet groove through the inlet opening, and passes through the particulate sensor to detect the particle concentration of the suspended particles contained in the air pollution, and the air pollution transported through the piezoelectric actuator is transported out of the outlet path defined by the gas-outlet groove through the ventilation hole, passes through the gas sensor for detecting, and then discharged from the gas-outlet of the base through the outlet opening.

10. The intelligent networked exhaust fan according to claim 9, wherein the laser component emits a parallel light source, and the parallel light source passes through the transparent window, wherein the particulate sensor detects suspended particulate and outputs the detection data.

11. The intelligent networked exhaust fan according to claim 9, wherein the at least one gas sensor comprises a temperature and humidity sensor, and the temperature and humidity sensor detects the temperature and humidity in air to output the detection data, wherein the at least one gas sensor comprises a volatile-organic-compound sensor, and the volatile-organic-compound sensor detects gas of carbon dioxide (CO₂) or volatile organic compounds (TVOC) to output the detection data, and the formaldehyde sensor detects gas of formaldehyde (HCHO) to output the detection data, wherein the at least one gas sensor comprises a bacteria sensor, and the bacteria sensor detects gas information of bacteria or fungi to output the detection data, wherein the at least one gas sensor comprises wherein the at least one gas sensor comprises a virus sensor, and the virus sensor detects gas of virus to output the detection data.

12. The intelligent networked exhaust fan according to claim 8, wherein the controlling circuit board comprises:
a power conversion component, providing DC voltage division modulation to output a required DC voltage, wherein the required DC voltage is transmitted through at least one connection interface to the gas detection main part for actuation operation and to the host driving controller for actuation operation;
a microcontroller (MCU), connected to the gas detection main part through the at least one connection interface to form the serial communication (IIC) signal for input, so that the detection data is calculated and analyzed, and connected through the at least one connection interface to output the Universal Asynchronous Transceiver and Transceiver (UART) signal and the General Purpose Input and Output (GP I/O) signal for regulation; and
a wireless communicator, receiving the detection data and transmitting to the networked cloud computing service device through external wireless communication, wherein the networked cloud computing service device collects, analyzes and monitors the detection data in real time and intelligently selects a control command, and the control command is received through the wireless communicator and transmitted to the microcontroller (MCU) to output the Universal Asynchronous Transceiver and Transceiver (UART) signal and the General Purpose Input and Output (GP I/O) signal for regulation of the host driving controller, so that the host driving controller is regulated to control the activation operation of the at least one air guiding fan, and dynamically adjust the operating frequency and the output air volume of the at least one air guiding fan.

13. The intelligent networked exhaust fan according to claim 12, further comprising a wired communication port, wherein the wired communication port is electrically connected to the controlling circuit board through a connection interface for external connection to a wired communication transmission, wherein the detection data is received and transmitted to the networked cloud computing service device through external wireless communication, the networked cloud computing service device collects, analyzes and monitors the detection data in real time and intelligently selects a control command, and the control command is received through the wired communication port and transmitted to the microcontroller (MCU) to output the Universal Asynchronous Transceiver and Transceiver (UART) signal and the General Purpose Input and Output (GP I/O) signal for regulation of the host driving controller, so that the host driving controller is regulated to control the activation operation of the at least one air guiding fan, and dynamically adjust the operating frequency and the output air volume of the at least one air guiding fan.

14. The intelligent networked exhaust fan according to claim 13, wherein the wired communication port is an RS485 port that communicates with the networked cloud computing service device through a wired line connection.

15. The intelligent networked exhaust fan according to claim 1, further comprising a filtering component disposed in the air guiding path, wherein the filtering component is a filter screen, and the filter screen is a filter screen with a minimum filtration efficiency value (MREV) equal to or greater than level 8.

16. The intelligent networked exhaust fan according to claim 15, wherein the filtering component is a filter screen, and the filter screen is a high-efficiency particulate air (HEPA) filter screen grade, wherein the high-efficiency particulate air (HEPA) filter screen is equal to or greater than a high-efficiency particulate air filter (HEPA) grade 10, with a dust holding capacity greater than 12,000 mg.

17. The intelligent networked exhaust fan according to claim 15, wherein the filtering component is a filter screen, and the filter screen is a ULPA14 filter screen grade, wherein the at least one filtering component is combined with a decomposition layer coated thereon to clean the air pollution through a chemical method of sterilization, the at least one filtering component is combined with a light irradiation element to clean the air pollution through a chemical method of sterilization, or the at least one filtering component is combined with a decomposition unit to clean the air pollution through a chemical method of sterilization.

18. The intelligent networked exhaust fan according to claim 17, wherein the decomposition layer is an activated carbon, and the activated carbon has a formaldehyde absorption capacity greater than 1500 mg, wherein the light irradiation element is a photo-catalyst unit including a photo catalyst and an ultraviolet lamp, or a photo-plasma unit including a nanometer irradiation tube, wherein the ultraviolet lamp has a power greater than 120 mW.

19. The intelligent networked exhaust fan according to claim 17, wherein the decomposition unit is a negative ion unit, a plasma ion unit or an electrostatic filtering unit.
